(19) United States
(12) Patent Application Publication
(10) Pub. No.: US 2017/0071579 A1
(43) Pub. Date: Mar. 16, 2017

(54) ULTRASOUND DIAGNOSTIC APPARATUS AND METHOD OF OPERATING THE SAME

(71) Applicant: SAMSUNG ELECTRONICS CO., LTD., Suwon-si (KR)

(72) Inventors: Hyun-woo KO, Yongin-si (KR); Jong-youb RYU, Suwon-si (KR); Hwan SHIM, Yongin-si (KR); Byeong-geun CHEON, Anyangsi (KR)

(73) Assignee: SAMSUNG ELECTRONICS CO., LTD., Suwon-si (KR)

(21) Appl. No.: 15/122,069

(22) PCT Filed: Feb. 25, 2015
(86) PCT No.: PCT/KR2015/001802

§ 371 Date: Aug. 26, 2016

(30) Foreign Application Priority Data
Feb. 28, 2014 (KR) 10-2014-0023799

(51) Int. Cl. A61B 8/00; A61B 8/14

(52) U.S. Cl. CPC A61B 8/54; A61B 8/14; A61B 8/56; A61B 8/4483; A61B 8/4254

(57) ABSTRACT

The ultrasound diagnostic apparatus includes an ultrasound transceiver that, by using a probe, transmits an ultrasound signal to an object, and receives an echo signal corresponding to the ultrasound signal from the object and an operation mode controller that sets an operation mode of the ultrasound transceiver to one of first and second operation modes, based on operation state information of the probe.

### TECHNICAL FIELD

One or more exemplary embodiments relate to an ultrasound diagnostic apparatus and a method of operating the same, and more particularly, to an ultrasound diagnostic apparatus and a method of operating the same, which reduce power consumption.

### BACKGROUND ART

Ultrasound diagnostic apparatuses transmit an ultrasound signal, generated by a transducer of a probe, onto an object and receive information of an echo signal reflected from the object, thereby obtaining an image of an internal region of the object. In particular, ultrasound diagnostic apparatuses are used for the medical purpose of observing the inside of an object, detecting a foreign material, and assessing an injury. Ultrasound diagnostic apparatuses display an image in real time, and are safe because there is no exposure to radiation, and thus are widely used.

Ultrasound diagnostic apparatuses may provide a brightness (B) mode in which a reflection coefficient of an ultrasound signal reflected from an object is shown as a two-dimensional (2D) image, a Doppler mode image in which an image of a moving object (particularly, blood flow) is shown by using the Doppler effect, and an elastic mode image in which a reaction difference between when compression is applied to an object and when compression is not applied to the object is expressed as an image.

### DISCLOSURE OF INVENTION

### Technical Problem

Power consumed by the ultrasound diagnostic apparatus and the amount of generated heat are increased for maintaining a quality of an image accquired by the ultrasound diagnostic apparatus.

### Solution to Problem

One or more exemplary embodiments include an ultrasound diagnostic apparatus and a method of operating the same, which reduce power consumption by using a low power mode and a normal mode, or drive some transducer elements to reduce power consumption, thereby minimizing a degradation in a quality of an image.

### Advantageous Effects of Invention

As described above, according to one or more of exemplary embodiments, the low power mode and the normal mode are automatically set and performed according to an operation state of the probe, and thus, power consumed by the ultrasound diagnostic apparatus is reduced, thereby decreasing the amount of generated heat.

The transducer elements are selectively driven by the aperture growth scheme or the sparse element scheme depending on a reception depth of an ultrasound signal, thereby minimizing a degradation in a quality of an image.

### BRIEF DESCRIPTION OF DRAWINGS

The above and/or other aspects will become more apparent by describing certain exemplary embodiments, with reference to the accompanying drawings, in which:

FIG. 1 is a block diagram illustrating a configuration of an ultrasound diagnostic apparatus according to an exemplary embodiment;

FIG. 2 is a block diagram illustrating a configuration of an ultrasound diagnostic apparatus according to an exemplary embodiment;

FIG. 3 is a flowchart illustrating a method of operating an ultrasound diagnostic apparatus according to an exemplary embodiment;

FIGS. 4A and 4B are diagrams for describing a low power mode operating method that reduces power by driving only some of a plurality of transducer elements according to an exemplary embodiment;

FIGS. 5A to 5B are diagrams for describing a low power mode operating method that reduces power by driving only some of a plurality of transducer elements according to an exemplary embodiment;

FIG. 6 is a flowchart illustrating a method of operating an ultrasound diagnostic apparatus according to an exemplary embodiment;

FIG. 7 is a flowchart illustrating a method of operating an ultrasound diagnostic apparatus according to an exemplary embodiment;

FIG. 8 is a diagram for describing the operating method of FIG. 7;

FIG. 9 is a flowchart illustrating a method of operating an ultrasound diagnostic apparatus according to an exemplary embodiment;

FIGS. 10A and 10B are diagrams for describing the operating method of FIG. 9;

FIGS. 11A and 11B are diagrams for describing the operating method of FIG. 9; and

FIGS. 12A, 12B, and 12C are ultrasonic images.

### BEST MODE FOR CARRYING OUT THE INVENTION

One or more exemplary embodiments include an ultrasound diagnostic apparatus and a method of operating the same, which reduce power consumption by using a low power mode and a normal mode, or drive some transducer elements to reduce power consumption, thereby minimizing a degradation in a quality of an image.

According to one or more exemplary embodiments, an ultrasound diagnostic apparatus includes: an ultrasound transceiver that, by using a probe, transmits an ultrasound signal to an object, and receives an echo signal corresponding to the ultrasound signal from the object; and an operation mode controller that sets an operation mode of the ultrasound transceiver to one of first and second operation modes, based on operation state information of the probe.

The first operation mode may be a low power mode, the second operation mode may be a normal mode, and the low power mode may be an operation mode that reduces at least one of a frequency of a transmitted or received ultrasound signal, a sampling rate of an analog-digital converter, number of channels, a frame rate of an ultrasound image, number of scan lines composing a frame image, and number of taps of an interpolation filter used in beamforming.

The ultrasound diagnostic apparatus may further include a sensor that senses a movement of the probe, wherein the operation mode controller may set the operation mode of the ultrasound transceiver to one of the first and second operation modes, based on movement information of the probe acquired from the sensor.

When a movement of the probe is greater than a predetermined value, the operation mode controller may allow the ultrasound transceiver to operate in the first operation mode, and when the movement of the probe is less than the predetermined value, the operation mode controller may allow the ultrasound transceiver to operate in the second operation mode.

The ultrasound diagnostic apparatus may further include an image generator that generates an ultrasound image, based on the received echo signal, wherein the operation mode controller may set the operation mode of the ultrasound transceiver to one of the first and second operation modes, based on the generated ultrasound image.

When it is determined that the probe operates in contact with the object, the operation mode controller may allow the ultrasound transceiver to operate in the second operation mode, and when it is determined that the probe operates in no contact with the object, the operation mode controller may allow the ultrasound transceiver to operate in the first operation mode.

When the probe transmits or receives the ultrasound signal to or from a predetermined region of interest, the operation mode controller may allow the ultrasound transceiver to operate in the second operation mode, and when the probe transmits or receives the ultrasound signal to or from a region instead of the predetermined region of interest, the operation mode controller may allow the ultrasound transceiver to operate in the first operation mode.

The ultrasound diagnostic apparatus may further include an input unit that receives a user input for setting the operation mode, wherein the operation mode controller may receive a user input which selects one of the first and second operation modes, and allow the ultrasound transceiver to operate in the selected operation mode.

The probe may include a plurality of transducer elements that transmit or receive the ultrasound signal, the ultrasound transceiver may include a plurality of analog front ends (AFEs) to which respective echo signals received from the plurality of transducer elements are input, number of the AFEs being less than number of the transducer elements, and the operation mode controller may include a first multiplexer that selects transducer elements, which are to be connected to the plurality of AFEs, from the plurality of transducer elements, based on a reception depth of the echo signal.

When the reception depth is less than a predetermined depth, the first multiplexer may select some continuously arranged transducer elements for the ultrasound transceiver to operate in the first operation mode, and when the reception depth is greater than the predetermined depth, the first multiplexer selects some transducer elements, in which an aperture size is maintained, for the ultrasound transceiver to operate in the second operation mode.

When the reception depth is less than the predetermined depth, the first multiplexer may select transducer elements which are continuously arranged with respect to a scan line from which an ultrasound signal is acquired.

When the reception depth is greater than the predetermined depth, the first multiplexer may select the transducer elements at the same intervals.

The operation mode controller may further include a second multiplexer that selects one of the first and second operation modes.

According to one or more exemplary embodiments, a method of operating an ultrasound diagnostic apparatus includes: acquiring operation state information of a probe; setting an operation mode of an ultrasound transceiver to one of first and second operation modes, based on the acquired operation state information of the probe; and transmitting an ultrasound signal to an object in the set operation mode, and receiving an echo signal corresponding to the ultrasound signal from the object.

The first operation mode may be a low power mode, the second operation mode may be a normal mode, and the low power mode may be an operation mode that reduces at least one of a frequency of a transmitted or received ultrasound signal, a sampling rate of an analog-digital converter, number of channels, a frame rate of an ultrasound image, number of scan lines composing a frame image, and number of taps of an interpolation filter used in beamforming.

The acquiring of the operation state information may include sensing a movement of the probe, and the setting of the operation mode may include setting the operation mode of the ultrasound transceiver to one of the first and second operation modes, based on movement information of the probe acquired from a sensor.

The setting of the operation mode may include: when a movement of the probe is greater than a predetermined value, setting the operation mode of the ultrasound transceiver to the first operation mode; and when the movement of the probe is less than the predetermined value, setting the operation mode of the ultrasound transceiver to the second operation mode.

The acquiring of the operation state information may include generating an ultrasound image, based on the received echo signal, and the setting of the operation mode may include setting the operation mode of the ultrasound transceiver to one of the first and second operation modes, based on the generated ultrasound image.

The setting of the operation mode may include: when it is determined that the probe operates in contact with the object, setting the operation mode of the ultrasound transceiver to the first operation mode, and when it is determined that the probe operates in no contact with the object, setting the operation mode of the ultrasound transceiver to the first operation mode.

The setting of the operation mode may include: when the probe transmits or receives the ultrasound signal to or from a predetermined region of interest, setting the operation mode of the ultrasound transceiver to the second operation mode; and when the probe transmits or receives the ultrasound signal to or from a region instead of the predetermined region of interest, setting the operation mode of the ultrasound transceiver to the first operation mode.

The method may further include receiving a user input for setting the operation mode, wherein the setting of the operation mode includes receiving a user input which selects one of the first and second operation modes, and setting the operation mode of the ultrasound transceiver to the selected operation mode.

The setting of the operation mode may include the ultrasound transceiver includes selecting some transducer elements, which are to be connected to a plurality of analog front ends, from a plurality of transducer elements, based on a reception depth of the echo signal.

The setting of the operation mode may include: when the reception depth is less than a predetermined depth, selecting some continuously arranged transducer elements for the ultrasound transceiver to operate in the first operation mode, and when the reception depth is greater than the predetermined depth, selecting some transducer elements, in which an aperture size is maintained, for the ultrasound transceiver to operate in the second operation mode.

The selecting of the some continuously arranged transducer elements may include selecting transducer elements which are continuously arranged with respect to a scan line from which the ultrasound signal is acquired.

The selecting of the some transducer elements may include selecting the transducer elements at the same intervals.

The setting of the operation mode may include selecting one of the first and second operation modes.

### Mode for the Invention

Certain exemplary embodiments are described in greater detail below with reference to the accompanying drawings.

In the following description, the same drawing reference numerals are used for the same elements even in different drawings. The matters defined in the description, such as detailed construction and elements, are provided to assist in a comprehensive understanding of exemplary embodiments. Thus, it is apparent that exemplary embodiments can be carried out without those specifically defined matters. Also, well-known functions or constructions are not described in detail since they would obscure exemplary embodiments with unnecessary detail.

Expressions such as “at least one of,” when preceding a list of elements, modify the entire list of elements and do not modify the individual elements of the list.

Also, when a part “includes” or “comprises” an element, unless there is a particular description contrary thereto, the part may further include other elements, not excluding the other elements. Terms such as “unit” and “module” described in specification denotes an element for performing at least one function or operation, and may be implemented in hardware, software or a combination of hardware and software.

The “ultrasound image” is an image of an object acquired by using an ultrasound wave. The “object” may include a person, an animal, a part of the person, or a part of the animal. For example, an object may include an organ such as a liver, a heart, a womb, a brain, breasts, an abdomen, or the like, or a blood vessel. Also, the term “object” may include a phantom. The phantom denotes a material having a volume that is very close to a density and effective atomic number of an organism, and may include a spherical phantom having a characteristic similar to a physical body.

The ultrasound image may be implemented in various ways. For example, the ultrasound image may be at least one of an amplitude (A) mode image, a brightness (B) mode image, a color (C) mode image, and a Doppler (D) mode image. Also, according to an exemplary embodiment, the ultrasound image may be a two-dimensional (2D) image or a three-dimensional (3D) image.

The term “user” used herein is a medical expert, and may be a doctor, a nurse, a medical technologist, a medical image expert, or the like, or may be an engineer who repairs a medical apparatus. However, the user is not limited thereto.

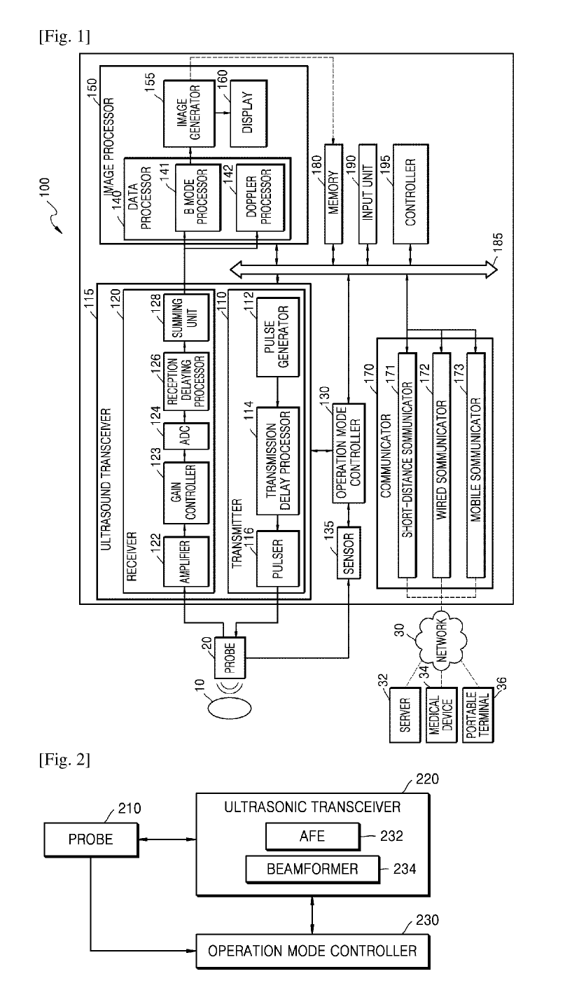

FIG. 1 is a block diagram illustrating a configuration of an ultrasound diagnostic apparatus 100 according to an exemplary embodiment. Referring to FIG. 1, the ultrasound diagnostic apparatus 100 according to an exemplary embodiment includes a probe 20, a sensor 135, an ultrasound transceiver 115, an operation mode controller 130, an image processor 150, a communicator 170, a memory 180, an input unit 190, and a controller 195. The above-described elements may be connected to each other through a bus 185.

The ultrasound diagnostic apparatus 100 may be implemented as a portable type as well as a card type. Examples of the portable diagnostic apparatuses may include picture archiving and communication system (PACS) viewers, smartphones, laptop computers, personal digital assistants (PDAs), tablet personal computers (PCs), etc., but are not limited thereto.

The probe 20 transmits ultrasound waves to an object 10 based on a driving signal applied by the ultrasound transceiver 115 and receives echo signals reflected by the object 10. The probe 20 includes a plurality of transducers, and the plurality of transducers oscillate based on electric signals transmitted thereto and generate acoustic energy, that is, ultrasound waves. Furthermore, the probe 20 may be connected to the main body of the ultrasound diagnostic apparatus 100 by wire or wirelessly. According to exemplary embodiments, the ultrasound diagnostic apparatus 100 may include a plurality of probes 20.

Examples of the sensor 135 may include an acceleration sensor, a gyro sensor, a tactile sensor, a proximity sensor, and a temperature sensor. The acceleration sensor is an element that converts a one-direction acceleration change into an electrical signal, and is being widely used with the advancement of micro-electromechanical system (MEMS) technology. Also, the gyro sensor is a sensor that measures an angular speed, and senses a direction twisted with respect to a reference direction.

The tactile sensor is a sensor that senses a touch by a specific object by a degree, in which a user feels, or more. The tactile sensor may sense various pieces of information such as a roughness of a touched surface, a stiffness of a touched object, a temperature of a touched point, etc.

The proximity sensor is a sensor that detects an object approaching a detection surface or an object near the detection surface by using an electromagnetic force or infrared light without any mechanical contact.

Examples of the proximity sensor include a transmissive photosensor, a directly reflective photosensor, a mirror reflective photosensor, a high frequency oscillation-type proximity sensor, a capacitive proximity sensor, a magnetic proximity sensor, and an infrared proximity sensor.

According to an exemplary embodiment, the sensor 135 may sense a moving speed of a probe, an angle at which the probe moves with respect to an object, a moving range of the probe, and whether the probe touches the object.

The operation mode controller 130 may sense an operation state of the probe 10, and set an operation mode based on the sensed operation state.

A transmitter 110 supplies a driving signal to the probe 20 and includes a pulse generator 112, a transmission delay processor 114, and a pulser 116. The pulse generator 112 generates pulses for forming transmission ultrasound waves based on a predetermined pulse repetition frequency (PRF), and the transmission delay processor 114 applies a delay time for determining transmission directionality to the pulses. Pulses to which a delay time is applied correspond to a plurality of piezoelectric vibrators included in the probe 20, respectively. The pulser 116 applies a driving signal (or a driving pulse) to the probe 20 as a timing corresponding to each pulse to which a delay time is applied.

A reception unit 120 generates ultrasound data by processing echo signals received from the probe 20 and may include an amplifier 122, a gain controller 123, an analog-digital converter (ADC) 124, a reception delay processor 126, and a summing unit 128. The amplifier 122 amplifies echo signals in each channel, and the ADC 124 analog-to-digital converts the amplified echo signals. The gain controller 123 adjusts a gain by using time gain compensation (TGC), in consideration that the echo signal is attenuated depending on a distance between a transducer and a reflector. The reception delay processor 126 applies delay times for determining reception directionality to the digital-converted echo signals, and the summing unit 128 generates ultrasound data by summing the echo signals processed by the reception delay processor 126. The ultrasound data is transferred to a data processor 140.

The image processor 150 generates an ultrasound image by scan-converting ultrasound data generated by the ultrasound transceiver 115 and displays the ultrasound image.

An ultrasound image may include a grayscale ultrasound image obtained by scanning an object in an amplitude (A) mode, a brightness (B) mode, a motion (M) mode, a blood flow Doppler image showing flow of blood (also referred to as a color Doppler image), a tissue Doppler image showing movement of tissues, and a spectral Doppler image showing moving speed of an object as a waveform.

A B mode processor 141 extracts B mode components from ultrasound data and processes the B mode components. An image generator 155 may generate an ultrasound image indicating signal intensities as brightness based on the extracted B mode components.

A Doppler processor 142 may extract Doppler components from ultrasound data, and the image generator 155 may generate a Doppler image indicating movement of an object as colors or waveforms based on the extracted Doppler components.

The image generator 155 according to an exemplary embodiment may generate a 2D ultrasound image via volume-rendering of volume data and may also generate an elasticity image which visualizes deformation of an object 10 due to pressure. Furthermore, the image generator 155 may display various additional information in an ultrasound image by using texts and graphics. The generated ultrasound image may be stored in the memory 180.

The display 160 displays the ultrasound image generated by the image generator 155. The display 160 may display various pieces of information processed by the ultrasound diagnostic apparatus 100, in addition to the ultrasound image, on a screen through a graphical user interface (GUI). The ultrasound diagnostic apparatus 100 may include two or more displays 160 depending on an implementation type.

The display 160 includes at least one of a liquid crystal display (LCD), a thin film transistor-liquid crystal display (TFT-LCD), an organic light-emitting diode (OLED), a flexible display, a 3D display, and an electrophoretic display.

Moreover, when the display 160 and the input unit 190 are implemented as a touch screen by forming a layer structure, the display 160 may be used as an input unit that enables information to be input by a user's touch, in addition to an output unit.

The touch screen may be configured to detect a touch pressure in addition to a touch input position and a touched area. Also, the touch screen may be configured to detect a proximity touch as well as a real touch.

The communicator 170 is connected to a network 30 in a wired or wireless manner to communicate with an external device or server. The communicator 170 may exchange data with a hospital server or a medical apparatus of a hospital which is connected thereto through a picture archiving and communication system (PACS). Also, the communicator 170 may perform data communication according to the digital imaging and communications in medicine (DICOM) standard.

The communicator 170 may transmit and receive data, such as an ultrasound image, ultrasound data, Doppler data, etc. of an object, associated with a diagnosis of the object over the network 30, and may also transmit and receive a medical image captured by a medical apparatus such as a computed tomography (CT) apparatus, a magnetic resonance imaging (MRI) apparatus, or an X-ray apparatus. Furthermore, the communicator 170 may receive information on a medical history or treatment schedule of a patient from a server, and use a diagnosis of an object. In addition, the communicator 170 may perform data communication with a portable terminal of a doctor or a patient, in addition to a server or medical apparatus of a hospital.

The communicator 170 may be connected to the network 30 in a wired or wireless manner, and may exchange data with a server 32, a medical apparatus 34, or a portable terminal 36. The communicator 170 may include one or more elements that enable communication with an external device, and for example, include a short-distance communicator 171, a wired communicator 172, and a mobile communicator 173.

The short-distance communicator 171 is a module for short-distance communication within a certain distance. Short-distance communication technology, according to an exemplary embodiment, may include wireless LAN, Wi-Fi, Bluetooth, Zigbee, Wi-Fi direct (WFD), ultra wideband (UWB), infrared data association (IrDA), Bluetooth low energy (BLE), and near field communication (NFC), but the short-distance communication technology is not limited thereto.

The wired communicator 172 is a module for communication using an electrical signal or an optical signal. Wired communication according to an exemplary embodiment may be implemented by a pair cable, a coaxial cable, an optical fiber cable, or an Ethernet cable.

The mobile communicator 173 transmits and receives a radio frequency (RF) signal to and from a base station, an external terminal, and a server over a mobile communication network. Here, the RF signal may include various types of data based on transmission and reception of a voice call signal, a video call signal, or a letter/multimedia message.

The memory 180 stores various pieces of information processed by the ultrasound diagnostic apparatus 100. For example, the memory 180 may store medical data, such as input/output ultrasound data and ultrasound images, associated with a diagnosis of an object, and may also store an algorithm or a program which is executed in the ultrasound diagnostic apparatus 100.

The memory 180 may be configured with various kinds of storage mediums such as a flash memory, a hard disk, an EEPROM, etc. Also, the ultrasound diagnostic apparatus 100 may operate web storage or a cloud server which performs a storage function of the memory 180 on a web.

The input unit 190 generates input data which is input by a user for controlling an operation of the ultrasound diagnostic apparatus 100. The input unit 190 may include hardware elements such as a keypad, a mouse, a touch pad, a trackball, a jog switch, but is not limited thereto. As another example, the input unit 190 may further include various sensors such as an electrocardiogram (ECG) measurement module, a breath measurement sensor, a voice recognition sensor, a gesture recognition sensor, a fingerprint recognition sensor, an iris recognition sensor, a depth sensor, a distance sensor, etc.

The input unit 190 may further include the touch screen in which the touch pad and the display 160 form the layer structure.

The ultrasound diagnostic apparatus 100 may display a specific mode ultrasound image and a control panel for an ultrasound image, on the touch screen. In addition, the ultrasound diagnostic apparatus 100 may sense a user's touch gesture for an ultrasound image through the touch screen.

The ultrasound diagnostic apparatus 100 according to an exemplary embodiment may physically include some buttons, frequently used by a user, from a plurality of buttons included in a control panel of general ultrasound diagnostic apparatuses, and the other buttons may be provided through a type of GUI on the touch screen.

The controller 195 controls an operation of the ultrasound diagnostic apparatus 100. That is, the controller 195 may control operations between the probe 20, the ultrasound transceiver 115, the image processor 150, the communicator 170, the memory 180, and the input unit 190 which are illustrated in FIG. 1.

Some or all of the probe 20, the ultrasound transceiver 115, the image processor 150, the communicator 170, the memory 180, the input unit 190, the operation mode controller 130, and the controller 195 may be operated by a software module, but are not limited thereto. Some of the above-described elements may be operated by a hardware module. Also, at least some of the ultrasound transceiver 115, the operation mode controller 130, the image processor 150, and the communicator 170 may be included in the controller 195, but are not limited to the implementation type.

FIG. 2 is a block diagram illustrating a configuration of a detail of an ultrasound diagnostic apparatus 200 according to an exemplary embodiment. The ultrasound diagnostic apparatus 200 may include a probe 210, an ultrasound transceiver 220, and an operation mode controller 230. For example, the ultrasound diagnostic apparatus 200 may be incorporated in the ultrasound diagnostic apparatus 100 and/or at least some of the probe 210, ultrasound transceiver 220, and operation mode controller 230 may correspond to the probe 20, ultrasound transceiver 115, and operation mode controller 130. Thus, the repeated descriptions will be omitted.

For example, the probe 210 of FIG. 1 is an element corresponding to the probe 20 of FIG. 1, and may include a plurality of transducer elements. Each of the transducer elements may transmit an ultrasound signal to an object according to a driving signal applied from the ultrasound transceiver 220, receive an echo signal reflected from the object, and establish a channel.

The ultrasound transceiver 220 of FIG. 2 is an element corresponding to the ultrasound transceiver 115 of FIG. 1, and may include an analog front end (AFE) 232 that receives an echo signal from each of the transducer elements. The AFE may include the amplifier 122, the gain controller 123, and the ADC 124 that are illustrated in and have been described above with reference to FIG. 1. The AFE may amplify the received echo signal, perform the TGC for correcting for attenuation caused by a depth of an ultrasound wave, and convert the echo signal into a digital radio frequency (RF) signal by using the ADC 124.

The ultrasound transceiver 220 may include a beamformer 234. The beamformer may include the reception delay processor 126 and the summing unit 128 which are illustrated in and have been described above with reference to FIG. 1. The digital RF signal obtained from the AFE is input to the beamformer, and the beamformer may store the digital RF signal by channel, and perform beamforming of the digital RF signal by using an interpolation beamforming scheme or a phase rotation beamforming scheme, thereby acquiring data beamformed by scan line.

The operation mode controller 230 may acquire operation state information of the probe 210, and set an operation mode of the ultrasound diagnostic apparatus 200 to a first operation mode or a second operation mode.

For example, the operation state information of the probe 210 may include information about an angle at which the probe 210 moves, displacement information about a movement of the probe 210, reception depth information of an echo signal received by the probe 210, information about whether the probe 210 touches an object, and information about a region in which the probe 210 transmits or receives an ultrasound signal.

When it is determined based on the operation state information of the probe 210 that the ultrasound diagnostic apparatus 200 needs to acquire a low-quality ultrasound image (when a precise diagnosis is not required), the operation mode controller 230 may set the operation mode to the first operation mode. The first operation mode is a low power mode, and may be a mode in which a quality of an acquired ultrasound image is degraded, but power consumed by the ultrasound diagnostic apparatus 200 is reduced.

On the other hand, when it is determined based on the operation state information of the probe 210 that the ultrasound diagnostic apparatus 200 needs to acquire a high-quality ultrasound image (when a precise diagnosis is required), the operation mode controller 230 may set the operation mode to the second operation mode. The second operation mode is a normal mode, and may be a mode in which power consumed by the ultrasound diagnostic apparatus 200 is increased, but a high-quality ultrasound image is acquired.

The operation mode controller 230 may include a first multiplexer. The multiplexer may select transducer elements, which are to be selected to the AFE, from a plurality of transducer elements on the basis of a reception depth of an echo signal.

For example, when the reception depth is less than a predetermined depth, the first multiplexer may select continuously arranged transducer elements from the plurality of transducer elements. However, when the reception depth is greater than the predetermined depth, the first multiplexer may select some transducer elements from the plurality of transducer elements so as to maintain an aperture size.

The operation mode controller 230 may further include a second multiplexer that selects the first and second operation modes.

The elements of the block diagrams of FIGS. 1 and 2 may be integrated, added, or omitted. That is, depending on the case, two or more elements may be integrated into one element, or one element may be subdivided into two or more elements.

FIG. 3 is a flowchart illustrating a method of operating an ultrasound diagnostic apparatus according to an exemplary embodiment.

Referring to FIG. 3, in operation S310, the ultrasound diagnostic apparatus 200 may acquire operation state information of a probe to determine an operation state of the probe.

The operation state information of the probe may include information about an angle at which the probe moves, displacement information about a movement of the probe, reception depth information of an echo signal received by the probe, information about whether the probe touches an object, and information about a region in which the probe transmits or receives an ultrasound signal.

In operation S320, the ultrasound diagnostic apparatus 200 may set an operation mode on the basis of the operation state information of the probe.

For example, when it is determined based on the operation state information of the probe that the ultrasound diagnostic apparatus 200 needs to acquire a low-quality ultrasound image (when a precise diagnosis is not required), the ultrasound diagnostic apparatus 200 may set the operation mode to the first operation mode. The first operation mode is the low power mode, and may be a mode in which a quality of an acquired ultrasound image is degraded, but power consumed by the ultrasound diagnostic apparatus 200 is reduced.

On the other hand, when it is determined based on the operation state information of the probe 210 that the ultrasound diagnostic apparatus 200 needs to acquire a high-quality ultrasound image (when a precise diagnosis is required), the operation mode controller 230 may set the operation mode to the second operation mode. The second operation mode is the normal mode, and may be a mode in which power consumed by the ultrasound diagnostic apparatus 200 increases, but a high-quality ultrasound image is acquired.

The ultrasound diagnostic apparatus 200 may transmit or receive an ultrasound wave in the set operation mode. For example, when the operation mode is set to the first operation mode, the ultrasound diagnostic apparatus 200 may operate in the low power mode.

When the ultrasound diagnostic apparatus 200 operates in the low power mode, the ultrasound diagnostic apparatus 200 may lower a frequency of a transmitted or received ultrasound wave, and lower a sampling rate of an analog to digital converter (ADC).

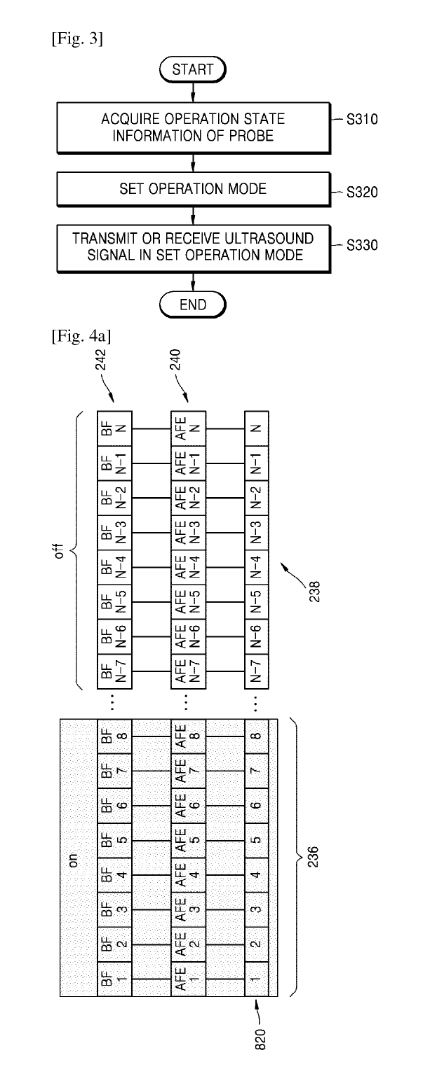

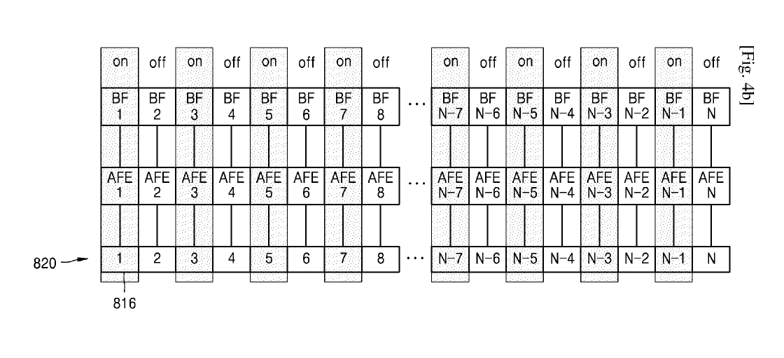

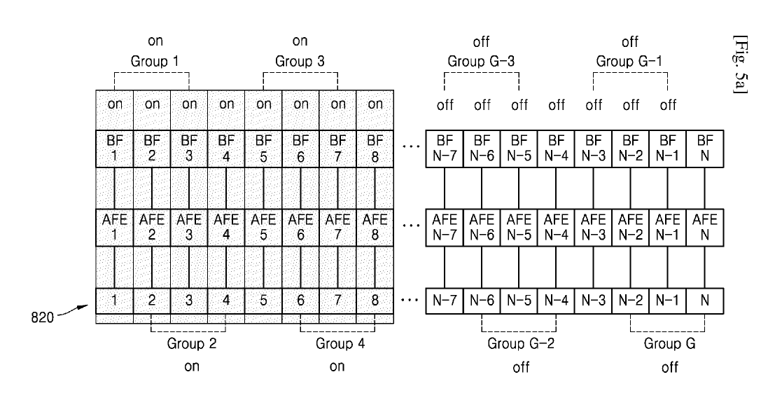

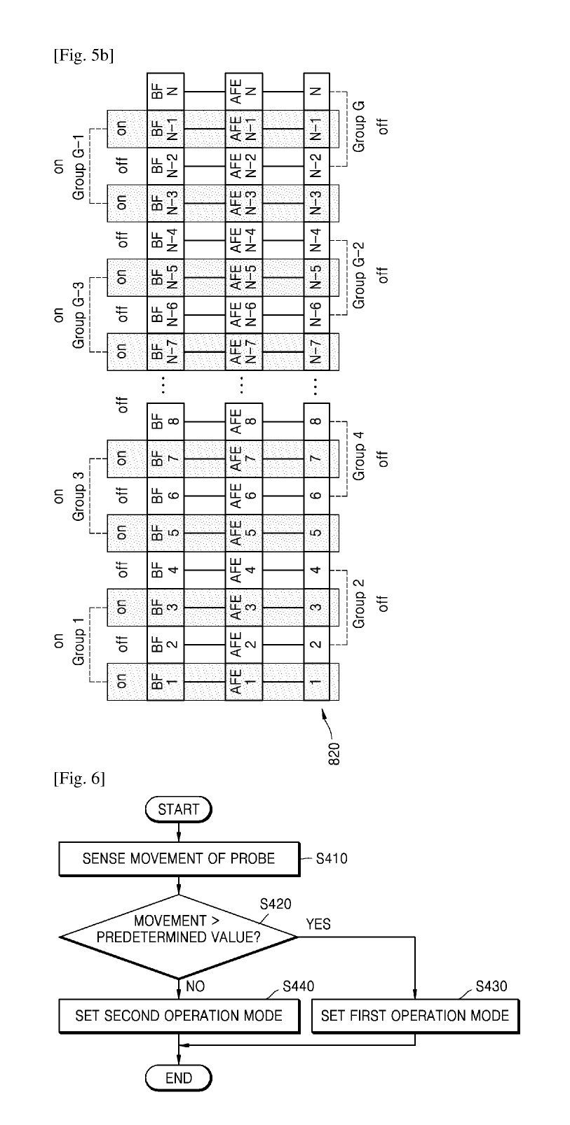

The ultrasound diagnostic apparatus 200 may reduce the number of channels. For example, the ultrasound diagnostic apparatus 200 does not drive some of a plurality of transducer elements included in the probe, and may cut off power of beamformers and AFEs corresponding to the undriven transducer elements. This will be described in detail with reference to FIGS. 4 and 5.

The ultrasound diagnostic apparatus 200 may increase a time interval, at which an ultrasound image is acquired, to decrease a frame rate of the ultrasound image, and cut off the power of the AFEs and beamformers at a time when an echo signal is not acquired.

The ultrasound diagnostic apparatus 200 may reduce the number of scan lines composing one frame image without any change in the frame rate of the ultrasound image, and cut off the power of the AFEs and beamformers at the time when the echo signal is not acquired.

The ultrasound diagnostic apparatus 200 may acquire the ultrasound image by using a plane wave imaging method. The ultrasound diagnostic apparatus 200 may decrease the frame rate to a low frame rate which corresponds to an imaging method based on a scan line, and cut off the power of the AFEs and beamformers at the time when the echo signal is not acquired.

The ultrasound diagnostic apparatus 200 may decrease the number of taps of an interpolation filter applied to a beamformer, or use a beamforming method that does not perform interpolation.

The ultrasound diagnostic apparatus 200 may lower a resolution of image data based on the echo signal, and transmit the image data.

On the other hand, when the ultrasound diagnostic apparatus 200 is set to the second operation mode, in order to acquire a high-quality ultrasound image, the ultrasound diagnostic apparatus 200 may maintain a frame rate, the number of scan lines composing one frame image, and the number of taps of the interpolation filter used in beamforming, or increase them, thereby transmitting or receiving an ultrasound signal.

FIGS. 4A, 4B, 5A, and 5B are diagrams for describing a low power mode operating method that reduces power by driving only some of a plurality of transducer elements according to an exemplary embodiment. The ultrasound diagnostic apparatus 200 may operate in the low power mode, and change a method that selects driven transducer elements depending on a reception depth of an echo signal so as to minimize a degradation in a quality of an ultrasound image.

FIG. 4A is a diagram illustrating a case in which the reception depth of the echo signal is less than a predetermined depth, and FIG. 4B is a diagram illustrating a case in which the reception depth of the echo signal is equal to or greater than the predetermined depth.

Referring to FIG. 4A, when the reception depth of the echo signal is less than the predetermined depth, the ultrasound diagnostic apparatus 200 may drive some continuously arranged transducer elements 236, and minimize a degradation in a quality of an image by using an aperture growth scheme that maintains a certain F number (a ratio of a reception depth and an aperture size).

The aperture growth scheme increases the number of continuously-driven transducer elements as a reception depth increases. When the aperture growth scheme is applied, the number of driven transducer elements may be expressed as the following Equation (1):

Number of elements=integer close to (dz*F number/dx) (1)

where dz is a reception depth, and dx is an interval between transducer elements. The number of driven transducer elements is calculated by using Equation (1), and the ultrasound diagnostic apparatus 200 may drive only the calculated number of the transducer elements, which are continuously arranged, from a plurality of transducer elements 820.

The ultrasound diagnostic apparatus 200 does not drive other transducer elements 238, and cuts off power of the AFEs and beamformers corresponding to the undriven transducer elements.

For example, a number of continuously arranged transducer elements is 1 to N. When a reception depth is less than a certain depth, the number of transducer elements calculated from Equation (1) may be 8, and an ultrasound signal for a fourth scan line is acquired, as illustrated in FIG. 4A, the ultrasound diagnostic apparatus 200 may drive a first element 1 to an eighth element 8 which are continuously arranged, i.e., a first element to an eighth element are turned on. The ultrasound diagnostic apparatus 200 does not drive the other transducer elements 238, i.e., a ninth element to an Nth element are turned off, and cuts off power of AFEs 240 and beamformers 242 corresponding to the other transducer elements 238.

When continuously driving the first element to the eighth element, an F number is maintained without any change. Accordingly, a degradation in a quality of an acquired ultrasound image is minimized, and since only eight elements are driven, power consumption is minimized.

Referring to FIG. 4B, when a reception depth of an echo signal is equal to or greater than a predetermined depth, a degradation in a quality of an image is minimized by using a sparse element scheme, which drives some transducer elements 816, without any change in an aperture size.

When some transducer elements are driven at the same intervals by using the sparse element scheme without any change in the aperture size, the some transducer elements are affected by a grating lobe. However, when a reception depth has a certain value or more compared to the aperture size, energy of an echo signal which is received at an angle corresponding to the grating lobe is reduced compared to energy of a main lobe, and thus, a degradation in a quality of an ultrasound image is not greatly affected.

For example, when the reception depth is a certain depth or more, with reference to FIG. 4B, the ultrasound diagnostic apparatus 200 may drive (turn on) elements 1, 3, 5, 7, . . . , N−3, and N−1, and does not drive (turn off) elements 2, 4, 6, . . . , N−2, and N. Therefore, the ultrasound diagnostic apparatus 200 cuts off power of AFEs and beamformers corresponding to the elements 2, 4, 6, . . . , N−2, and N.

In comparison with a case in which all transducer elements are driven (N number of transducer elements), as described above, when driving the elements 1, 3, 5, 7, . . . , N−3, and N−1, the aperture size is maintained, and thus, a degradation in a quality of an ultrasound image is minimized. Also, since only half (N/2 number of elements) of the transducer elements is driven, power consumption is minimized. Also, as described above, since the reception depth is a certain depth or more, an influence of the grating lobe is small.

As illustrated in FIGS. 4A and 4B, a method of selecting a transducer element is differently applied depending on a reception depth of an echo signal, and the turn-on/off of a plurality of transducer elements is separately controlled.

The ultrasound diagnostic apparatus 200 may group a plurality of transducer elements, and control the transducer elements by group, thereby further decreasing power consumption.

FIGS. 5A and 5B are diagrams for describing a method in which a plurality of transducer elements are grouped, and are controlled by group.

Referring to FIGS. 5A and 5B, an N number of transducer elements is divided into G number of groups. Elements 1 and 3 may be set as a first group “Group 1”, elements 2 and 4 may be set as a second group “Group 2”, elements 5 and 7 may be set as a third group “Group 3”, elements 6 and 8 may be set as a fourth group “Group 4”, elements N−7 and N−5 may be set as a (G-3) group “Group G-3”, elements (N−6) and (N−4) may be set as a (G-2) group “Group G-2”, elements (N−3) and (N−1) may be set as a (G-1) group “Group G-1”, and elements (N−2) and N may be set as a Gth group “Group G”.

When the plurality of transducer elements are grouped, as illustrated in FIG. 5A, the ultrasound diagnostic apparatus 200 may drive the first to fourth groups to drive eight continuous elements, without separately controlling the eight elements. Accordingly, power consumption for controlling a plurality of transducer elements is reduced.

Moreover, as illustrated in FIG. 5B, the ultrasound diagnostic apparatus 200 may drive the first, third, . . . , (G-3), and (G-1) groups to drive N/2 number of elements, without separately controlling the N/2 elements. Accordingly, power consumption for controlling a plurality of transducer elements is reduced.

FIG. 6 is a flowchart illustrating a method of operating an ultrasound diagnostic apparatus according to an exemplary embodiment.

Referring to FIG. 6, in operation S410, the ultrasound diagnostic apparatus 200 may sense a movement of a probe. The ultrasound diagnostic apparatus 200 may include a sensor or sensors including at least one of an acceleration sensor, a gyro sensor, a proximity sensor, a tactile sensor, and a temperature sensor. The ultrasound diagnostic apparatus 200 may sense the movement of the probe by using the sensors.

For example, the ultrasound diagnostic apparatus 200 may sense a moving speed of a probe, an angle at which the probe moves with respect to an object, a moving range of the probe, and whether the probe touches the object.

In operation S420, the ultrasound diagnostic apparatus 200 compares data about the movement of the probe and a predetermined value. When the movement of the probe is greater than the predetermined value, the ultrasound diagnostic apparatus 200 may set the operation mode to the first operation mode. On the other hand, when the movement of the probe is less than or equal to the predetermined value, the ultrasound diagnostic apparatus 200 may set the operation mode to the second operation mode.

For example, by using the sensor, the ultrasound diagnostic apparatus 200 may measure a distance by which the probe moves, and compare the measured distance and a predetermined value. Also, the ultrasound diagnostic apparatus 200 may measure an angle at which the probe moves, and compare the measured angle and the predetermined value. In addition, the ultrasound diagnostic apparatus 200 may measure a speed at which the probe moves, and compare the measured speed and the predetermined value.

When the movement of the probe is large as in a case where at least one of the measured distance, angle, and speed is greater than the predetermined value, the ultrasound diagnostic apparatus 200 may determine the probe as moving to locate a region which is to be diagnosed.

Therefore, the ultrasound diagnostic apparatus 200 may set the operation mode to the first operation mode in operation S430, and as described above with reference to FIG. 3, may transmit or receive an ultrasound wave in the low power mode.

On the other hand, when the movement of the probe is small as in a case where at least one of the measured distance, angle, and speed is less than or equal to the predetermined value, the ultrasound diagnostic apparatus 200 may determine the probe as moving for a precise diagnosis. Therefore, the ultrasound diagnostic apparatus 200 may set the operation mode to the second operation mode in operation S440, and as described above with reference to FIG. 3, may transmit or receive an ultrasound wave in the normal mode.

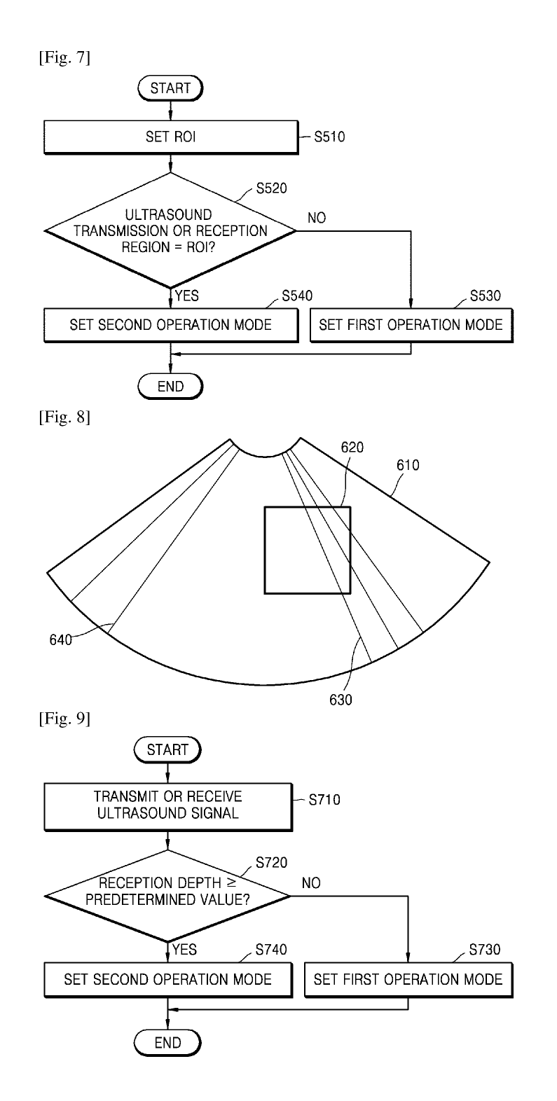

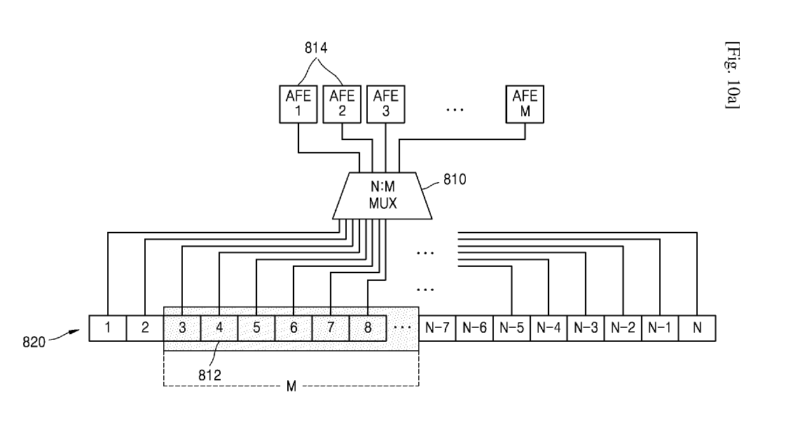

FIG. 7 is a flowchart illustrating a method of operating an ultrasound diagnostic apparatus according to an exemplary embodiment. FIG. 8 is a diagram for describing the operating method of FIG. 7.

In operation S510, the ultrasound diagnostic apparatus 200 may set a region of interest (ROI). For example, by using a mouse or a touch screen, the ultrasound diagnostic apparatus 200 may set the ROI on the basis of a user input which selects the ROI. Alternatively, the ultrasound diagnostic apparatus 200 may set the ROI by using an eyeball mouse, a method of measuring an eyeball position and look direction of a user, or a probe. However, an exemplary embodiment is not limited thereto, and the ultrasound diagnostic apparatus 200 may set the ROI by using various methods.

In operation S520, the ultrasound diagnostic apparatus 200 may determine whether a region which the probe transmits or receives an ultrasound wave to or from is a predetermined ROI 620. For example, as illustrated in FIG. 8, the ultrasound diagnostic apparatus 200 may determine whether scan lines 630 and 640 transmitting or receiving the ultrasound wave are included in the predetermined ROI 620 of an image 610.

When the scan line 630 which the probe transmits or receives the ultrasound wave to or from is included in the predetermined ROI 620, the ultrasound diagnostic apparatus 200 may set the operation mode to the second operation mode (operation S540), and, as described above with reference to FIG. 3, may transmit or receive the ultrasound wave in the normal mode.

On the other hand, when the scan line 640 which the probe transmits or receives the ultrasound wave to or from is not included in the predetermined ROI 620, the ultrasound diagnostic apparatus 200 may set the operation mode to the first operation mode (operation S530), and, as described above with reference to FIG. 3, may transmit or receive the ultrasound wave in the low power mode.

In addition, the ultrasound diagnostic apparatus 200 may determine an operation state of the probe on the basis of an acquired ultrasound image.

For example, when the probe does not touch an object, the ultrasound diagnostic apparatus 200 may acquire an ultrasound signal from only a surface of the object because an impedance mismatch between transducer elements and an air layer is large, and since it is unable to acquire an ultrasound signal from the other region, the ultrasound diagnostic apparatus 200 may display only an ultrasound image of a region corresponding to the surface without displaying an ultrasound image of the other region. Therefore, the ultrasound diagnostic apparatus 200 may analyze the ultrasound image to determine whether the probe touches the object.

The ultrasound diagnostic apparatus 200 may sense a change between the ultrasound image and a frame. When the change is large, the ultrasound diagnostic apparatus 200 may determine a movement of the probe as being large, and when the change is small, the ultrasound diagnostic apparatus 200 may determine the movement of the probe as being small.

Therefore, when the probe does not touch the object or the movement of the probe is large, the ultrasound diagnostic apparatus 200 may set the operation mode to the first operation mode, and, as described above with reference to FIG. 3, the ultrasound diagnostic apparatus 200 may transmit or receive an ultrasound wave in the low power mode.

On the other hand, when the probe touches the object and the movement of the probe is small, the ultrasound diagnostic apparatus 200 may set the operation mode to the second operation mode, and, as described above with reference to FIG. 3, the ultrasound diagnostic apparatus 200 may transmit or receive the ultrasound wave in the normal mode.

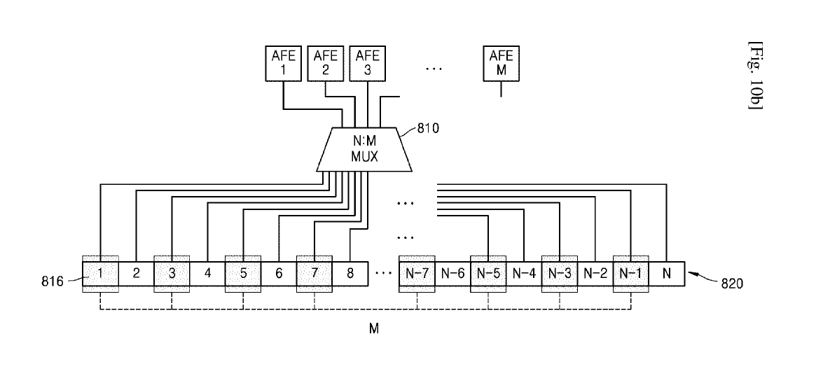

FIG. 9 is a flowchart illustrating a method of operating an ultrasound diagnostic apparatus according to an exemplary embodiment. FIGS. 10A to 12 are diagrams for describing the operating method of FIG. 9.

Referring to FIG. 10A, the ultrasound diagnostic apparatus 200 according to an exemplary embodiment may include N number of transducer elements and M number of AFEs. The ultrasound diagnostic apparatus 200 may selectively drive only some of the N transducer elements, for decreasing power consumption. Therefore, the ultrasound diagnostic apparatus 200 may include a multiplexer 810 that selects some of the N transducer elements, and the multiplexer 810 may be an N:M multiplexer based on the number (N) of transducer elements and the number (M) of AFEs.

Referring to FIG. 9, in operation S710, the ultrasound diagnostic apparatus 200 may transmit an ultrasound wave to an object, and receive a reflected echo signal.

The ultrasound diagnostic apparatus 200 may operate in the first or second operation mode depending on a reception depth of an ultrasound signal.

For example, in operation S720, the ultrasound diagnostic apparatus 200 may compare the reception depth of the ultrasound signal and a predetermined depth. When the reception depth is less than the predetermined depth, the multiplexer 810 may select some transducer elements 812 according to the aperture growth scheme which has been described above with reference to FIG. 4A. The operation mode may be set to the first operation mode in operation S730.

The multiplexer 810 may select the M transducer elements 812 which are continuously arranged with respect to a scan line from which an ultrasound signal is acquired. Also, when the number of transducer elements calculated from Equation (1) is less than M, the ultrasound diagnostic apparatus 200 may cut off power of some of the AFEs 814 receiving the echo signal and power of beamformers corresponding to the some AFEs.

When the reception depth of the ultrasound signal is greater than or equal to the predetermined depth, as illustrated in FIG. 10B, the multiplexer 810 may select some transducer elements according to the sparse element scheme which has been described above with reference to FIG. 4B. The operation mode may be set to the second operation mode in operation S740.

In this case, the multiplexer 810 maintains an aperture size, and may select M transducer elements 816 from the N transducer elements 820. The multiplexer 810 may select the M transducer elements at the same intervals, for example, every other transducer element.

For example, when the number (M) of AFEs is half of the number (N) of transducer elements, the multiplexer 810 may select elements 1, 3, 5, 7, . . . , N−3, and N−1, or may select elements 2, 4, 6, 8, . . . , N−2, and N. When the number (M) of AFEs is one-third of the number (N) of transducer elements, the multiplexer 810 may select elements 1, 4, 7, . . . , and N−2, e.g., every third transducer element.

As described above, the ultrasound diagnostic apparatus 200 may include AFEs less than the number of transducer elements, thereby decreasing power consumption. When the reception depth is less than the predetermined depth, the ultrasound diagnostic apparatus 200 may operate in the first operation mode based on the aperture growth scheme, and thus minimizes a degradation in a quality of an ultrasound image. On the other hand, when the reception depth is equal to or greater than the predetermined depth, the ultrasound diagnostic apparatus 200 may operate in the second operation mode based on the sparse element scheme, and thus minimizes the degradation in the quality of the ultrasound image.

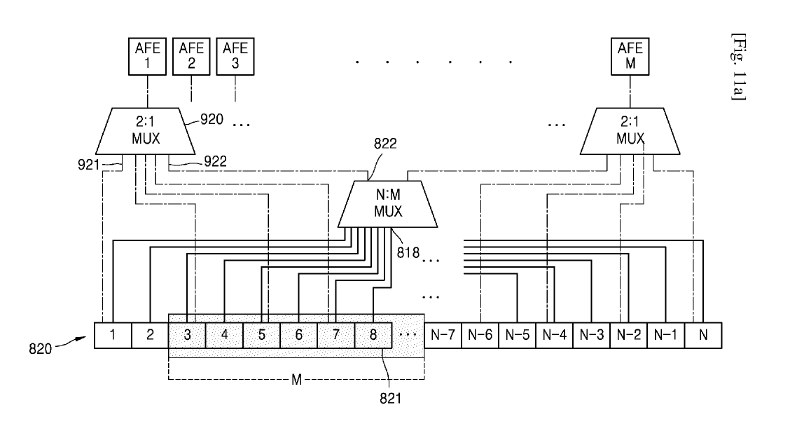

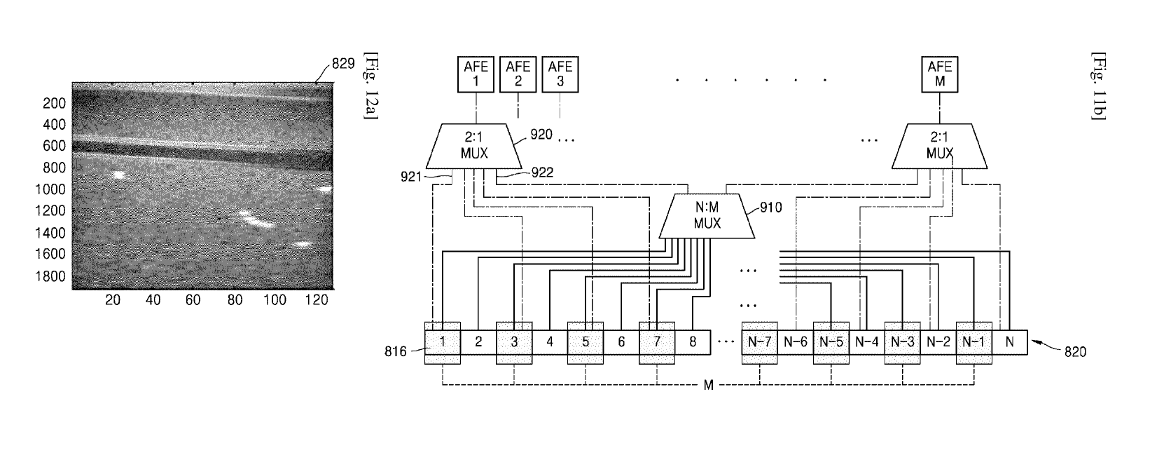

FIGS. 11A and 11B are diagrams illustrating a configuration of an ultrasound diagnostic apparatus according to an exemplary embodiment.

Referring to FIGS. 11A and 11B, the ultrasound diagnostic apparatus 200 may include M number of 2:1 multiplexers, i.e., one or more of second multiplexers 920 and one N:M multiplexer, i.e., a first multiplexer 910. Alternatively, instead of the M number of 2:1 second multiplexers, one integrated multiplexer may be used, but this is not limiting.

Input terminals 818 of the first multiplexer 910 may be connected to N number of transducer elements 820, and the first multiplexer 910 may select M number of transducer elements 812 from the N transducer elements 820 according to the first operation mode based on the aperture growth scheme. For example, as described above with reference to FIG. 4, the first multiplexer 910 may select the M transducer elements with respect to a scan line from which an ultrasound signal is acquired.

The second multiplexer 920 may select one of the first and second operation modes. First input terminals 921 of a plurality of the second multiplexers 920 may be respectively connected to the M transducer elements 816 of the N transducer elements 820 which are selected according to the second operation mode based on the sparse element scheme, and second input terminals 922 of the plurality of second multiplexers 920 may be respectively connected to output terminals 822 of the first multiplexer 910.

For example, when M is N/2, the first input terminals 921 of M number of the second multiplexers 920 may be respectively connected to elements 1, 3, 5, 7, . . . , N−7, N−5, N−3, and N−1, and the second input terminals 922 of the M second multiplexers 920 may be respectively connected to the output terminals 822 of the first multiplexer 910.

Therefore, when the reception depth is less than the predetermined depth, the second multiplexer 920 may select a first value (corresponding to selection of the first operation mode), and when the reception depth is equal to or greater than the predetermined depth, the second multiplexer 920 may select a second value (corresponding to selection of the second operation mode) and output a first value or a second value through the first input terminal 921.

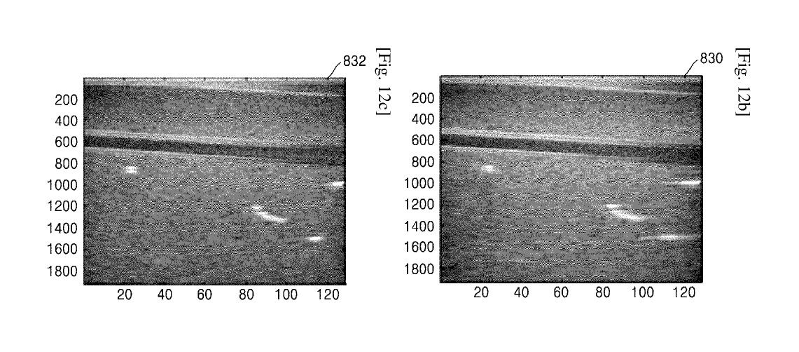

FIG. 12A shows an ultrasound image 829 acquired by using 128 channels. FIG. 12B shows an ultrasound image 830 acquired by using 64 channels which are continuous with respect to a scan line, irrespective of a reception depth. FIG. 12C shows an ultrasound image 832 which is acquired by using the first operation mode (channel selection based on the aperture growth scheme) and the second operation mode (channel selection based on the sparse element scheme, and selection of elements from 128 elements at three-element intervals), based on the reception depth.

Comparing FIGS. 12A and 12B, a quality of an ultrasound image 830 is degraded in a region where the reception depth is deep. Comparing FIGS. 12A and 12C, however, the quality of the ultrasound image 832 is hardly degraded even in the region where the reception depth is deep.

As described above, according to one or more of exemplary embodiments, the low power mode and the normal mode are automatically set and performed according to an operation state of the probe, and thus, power consumed by the ultrasound diagnostic apparatus is reduced, thereby decreasing the amount of generated heat.

The transducer elements are selectively driven by the aperture growth scheme or the sparse element scheme depending on a reception depth of an ultrasound signal, thereby minimizing a degradation in a quality of an image.

The elements of the ultrasound diagnostic apparatus and the operating method thereof according to exemplary embodiments may also be embodied as computer-readable codes on a computer-readable recording medium. The computer-readable recording medium is any data storage device that can store data which can be thereafter read by a computer system. Examples of the computer-readable recording medium include read-only memory (ROM), random-access memory (RAM), CD-ROMs, magnetic tapes, floppy disks, and optical data storage. The computer-readable recording medium can also be distributed over network coupled computer systems so that the computer readable code may be stored and executed in a distributed fashion.

The described-above exemplary embodiments and advantages are merely exemplary and are not to be construed as limiting. The present teaching can be readily applied to other types of apparatuses. The description of exemplary embodiments is intended to be illustrative, and not to limit the scope of the claims, and many alternatives, modifications, and variations will be apparent to those skilled in the art.

1. An ultrasound diagnostic apparatus comprising:
an ultrasound transceiver configured to transmit an ultrasound signal to an object by using a probe, and receive an echo signal corresponding to the ultrasound signal from the object; and
an operation mode controller configured to set an operation mode of the ultrasound transceiver to one of a first operation mode and a second operation mode, based on operation state information of the probe.

2. The ultrasound diagnostic apparatus of claim 1, wherein the first operation mode is a low power mode that is configured to reduce at least one of a frequency of a transmitted or received ultrasound signal, a sampling rate of an analog to digital converter, a number of channels, a frame rate of an ultrasound image, a number of scan lines composing a frame image, and a number of taps of an interpolation filter used in beamforming, and
the second operation mode is a normal mode which uses an increased power as compared to the low power mode.

3. The ultrasound diagnostic apparatus of claim 1, further comprising a sensor configured to sense a movement of the probe,
wherein the operation mode controller is configured to set the operation mode of the ultrasound transceiver to one of the first operation mode and the second operation mode based on a movement value of the probe acquired from the sensor.

4. The ultrasound diagnostic apparatus of claim 1, wherein the operation mode controller is configured to set the ultrasound transceiver to operate in the first operation mode, in response to the movement value of the probe being greater than a predetermined value, and to set the ultrasound transceiver to operate in the second operation mode, in response to the movement value of the probe being less than or equal to the predetermined value.

5. The ultrasound diagnostic apparatus of claim 1, further comprising an image generator configured to generate an ultrasound image based on the received echo signal,
wherein the operation mode controller is configured to set the operation mode of the ultrasound transceiver to one of the first operation mode and the second operation mode based on the generated ultrasound image.

6. The ultrasound diagnostic apparatus of claim 1, wherein the operation mode controller is configured to set the ultrasound transceiver to operate in the second operation mode in response to determining that the probe operates in contact with the object, and to set the ultrasound transceiver to operate in the first operation mode in response to determining that the probe does not have a contact with the object.

7. The ultrasound diagnostic apparatus of claim 1, wherein the operation mode controller is configured to set the ultrasound transceiver to operate in the second operation mode in response to the probe transmitting or receiving the ultrasound signal to or from a region of interest (ROI) of the object, and to set the ultrasound transceiver to operate in the first operation mode in response to the probe transmitting or receiving the ultrasound signal to or from a region different from the ROI.

8. The ultrasound diagnostic apparatus of claim 1, further comprising an input unit configured to receive a user input,
wherein the operation mode controller is configured to receive, from the input unit, the user input which selects one of the first operation mode and the second operation mode, and set the ultrasound transceiver to operate in the selected operation mode.

9. An ultrasound imaging method comprising:
acquiring operation state information of a probe;
setting an operation mode of an ultrasound transceiver to one of a first operation mode and a second operation mode, based on the acquired operation state information of the probe; and
transmitting an ultrasound signal to an object in the set operation mode, and receiving an echo signal corresponding to the ultrasound signal from the object.

10. The method of claim 9, wherein the first operation mode is a low power mode that is configured to reduce at least one of a frequency of a transmitted or received ultrasound signal, a sampling rate of an analog to digital converter, a number of channels, a frame rate of an ultrasound image, a number of scan lines composing a frame image, and a number of taps of an interpolation filter used in beamforming, and
the second operation mode is a normal mode which uses an increased power as compared to the low power mode.

11. The method of claim 9, wherein the acquiring the operation state information comprises sensing a movement of the probe, and
the setting the operation mode comprises setting the operation mode of the ultrasound transceiver to one of the first operation mode and the second operation mode based on a movement value of the probe acquired from a sensor.

12. The method of claim 9, wherein the setting the operation mode comprises:
setting the operation mode of the ultrasound transceiver to the first operation mode in response to the movement value of the probe being greater than a predetermined value; and
setting the operation mode of the ultrasound transceiver to the second operation mode in response to the movement value of the probe being less than or equal to the predetermined value.

13. The method of claim 9, wherein the acquiring the operation state information comprises generating an ultrasound image based on the received echo signal, and
the setting the operation mode comprises setting the operation mode of the ultrasound transceiver to one of the first operation mode and the second operation mode based on the generated ultrasound image.

14. The method of claim 9, wherein the setting the operation mode comprises:
setting the operation mode of the ultrasound transceiver to the first operation mode in response to determining that the probe operates in contact with the object, and
setting the operation mode of the ultrasound transceiver to the first operation mode in response to determining that the probe operates in no contact with the object.

15. The method of claim 9, wherein the setting the operation mode comprises:
setting the operation mode of the ultrasound transceiver to the second operation mode in response to the probe transmitting or receiving the ultrasound signal to or from a region of interest (ROI) of the object; and
setting the operation mode of the ultrasound transceiver to the first operation mode in response to the probe transmitting or receiving the ultrasound signal to or from a region different from the ROI.
